(19) United States
(12) Patent Application Publication
(10) Pub. No.: US 2005/0106224 A1
(43) Pub. Date: May 19, 2005

(54) Wound care device

(76) Inventor: Michael H. Qvist, Kobenhavn O (DK)

Correspondence Address: JACOBSON HOLMAN PLLC, 400 SEVENTH STREET N.W., SUITE 600, WASHINGTON, DC, 20004, US

(21) Appl. No.: 10/953,068

(22) Filed: Sep. 30, 2004

(30) Foreign Application Priority Data
Sep. 30, 2003 (DK) PA 2003 01427

(51) Int. Cl. A61K 38/48; A61K 38/46; A61K 9/14

(52) U.S. Cl. 424/445; 424/94.64; 424/94.65

(57) ABSTRACT

A wound care device comprising a container having two or more separate compartments, a first compartment containing a first component, a second compartment containing a second component, wherein the container comprises means for bringing the first component into direct contact with the second component. The device may be suitable for wound care products such as hydrogels comprising components being unstable in each other's presence.

### BACKGROUND OF THE INVENTION

1. Field of the Invention

The invention relates to a wound care device, especially a device for preparing a medicated hydrogel.

2. Description of the Related Art

Wound care devices comprising active ingredients for release to a skin or wound site are well known in the art. Such devices may be in the form of adhesive dressings or patches with e.g. a drug incorporated therein. The drug may be released and activated when brought into contact with wound exudates. Active ingredients may often comprise compounds that are sensitive to moisture. This may give rise to problems when preparing wound care devices comprising a higher degree of moisture, such as hydrogels, pastes or creams as stability problems may arise. Many active or therapeutic agents may decompose or become inactive when exposed to moisture for longer periods, e.g. during storage of the product. Attempts have been made to solve these problems e.g. by encapsulating or immobilizing the active agent. However, this may be complicated and expensive and add extra steps to the manufacturing process. Furthermore, it may prevent the active component to reach the therapeutic target.

Containers for mixing two or more components prior to use are known, e.g. from hair dyes and two-component adhesives, but also medical devices.

U.S. Pat. No. 5,071,034 discloses a liquid distributing device comprising a receptacle, an intermediate part and an upper part. The intermediate part has the shape of a bellow and serves as a mixing chamber. The upper part may contain a liquid and the receptacle may contain a liquid or a dry substance. Before use, the membranes separating the upper part and the receptacle from the intermediate part are ruptured, and by shaking the device the contents of the two parts are combined. In order to ensure proper mixing, at least one of the components is a liquid.

From U.S. Pat. No. 5,928,213 is known a flexible container with multiple compartments, separated by peelable seals. The container is for liquids, and manipulating the container, thus breaking the seals between the compartments, combines the contents. Shaking the container mixes the components.

U.S. patent application No. 2002/0040739 discloses a flexible mixing chamber. Receptacles, containing the components to be mixed, are attached to the mixing chamber; the contents are inserted into the chamber, and mixed by manipulating the chamber. The construction demands multiple receptacles, which may give rise to leakage and contamination of the components.

European patent application No. 395 758 discloses a separate storage container. The container comprises two separate chambers and a piercing needle, which may be activated in order to break the seal between the two containers. Preferably, one chamber contains a solvent and another chamber a liquid or powderous component to be combined with the solvent. The container would not be applicable for viscous components such as hydrogels, as proper mixing will be difficult.

None of the above-mentioned references are suitable for use for hydrogels. Hydrogels has a high content of water, which makes it difficult to incorporate and store components being sensible to moisture. Furthermore, hydrogels are usually viscous which renders mixing with other components homogeneously difficult.

Thus there is still a need for a wound care device comprising a hydrogel and at least one second component being capable of in a simple manner mixing the components before use.

### SUMMARY OF THE INVENTION

The object of the invention is to provide a wound care device comprising moisture sensitive or unstable active ingredients, said device showing high stability during storage.

Another object of the present invention is to provide a wound care device, which contains multiple components that may be unstable when combined, but stable when kept in separate compartments.

A third object of the invention is to provide a multi-component wound care device, which is easy to handle and apply.

A fourth object of the present invention is to provide a medicated hydrogel that is stable during storage.

A fifth abject of the invention is to provide a medicated hydrogel with a high relative activity of enzyme.

### BRIEF DESCRIPTION OF THE DRAWINGS

The invention is disclosed more in detail with reference to the drawings in which

FIG. 1 shows one embodiment of the invention,

FIG. 2 shows another embodiment of the invention,

FIG. 3 shows a third embodiment of the invention and

FIG. 4 shows a fourth embodiment of the invention.

FIGS. 5a and 5b shows a fifth embodiment of the invention.

### DETAILED DESCRIPTION OF THE PRESENT INVENTION

The invention relates to a wound care device comprising a container having two or more separate compartments, a first compartment containing a first component, a second compartment containing a second component, wherein the container comprises means for bringing the first component into direct contact with the second component and means for mixing the components, wherein the first component is a hydrogel.

The device according to the present invention renders it possible to prepare a wound care device comprising one or more components, which may be unstable in the presence of one or more of the other components of the device.

Because the components of the device are mixed immediately or shortly before use, no additional encapsulation or formulation of the active ingredient(s) in order to protect it from the moisture may be needed. However, encapsulation or other formulation of the active ingredient in order to provide a controlled or sustained release of the agent may be provided.

When preparing medicated wound care devices, especially hydrogels, it is often a problem that the active ingredient is not stable when exposed to aqueous environment for longer periods, such as during storage. Examples may be silver-compounds, which may be discolored during storage, proteins which may become degraded due to water-dependent degradation reactions, enzymes which may become inactivated, e.g. due to autolysis or aggressive components, which may decompose the hydrogel of the device.

In the device of the present invention, the active ingredient is not exposed to aqueous environment before use, as the agent is not directly embedded or soluted in the hydrogel during storage, but kept in separate compartments. Thus, the agent will remain stable during storage.

In the device of the invention the first and the second component are separated in different compartments of the container. The components are separated by appropriate means such as a membrane that breaks under pressure or by penetration of a spear, or the means may be prepared from glassy material that can be broken by a snap.

Before use of the device the first and the second component are mixed together. The mixing of the components may be completely homogenous or it may be more roughly blended, but still united.

The device of the present invention may be easily handled. The blending of the components of the device may be carried out in a way where only one hand is needed for the process. Alternatively, two hands may be needed for the activation and blending, but it is preferred that the united components of the device may be applied to the desired body part by use of one hand only. Thus it is only when mixing the first component with the active sensible component that two hands may be needed while application of the united components to the wound may easily be done with one hand.

This mixing process may begin when the components are combined, e.g. by manipulating the container or it may be done mechanically, e.g. when the contents of the device are brought out of the device such as by squeezing. During the mixing process the components are blended substantially homogeneously, thus providing a hydrogel with a substantially no variation in the content of active ingredient when applied.

The first component of the device of the invention is a hydrogel. The hydrogel may comprise one or more biologically or pharmaceutically active ingredients, such as odor controllers, antibiotics, antiseptics, such as silver compounds, anesthetics or analgesics for pain relief.

In one embodiment of the invention the hydrogel comprises a pain-killing agent, such as ibuprofen, ketoprofen, flurbiprofen, acetylsalicylic acid, salicylic acid, diclofenac, lornoxicam, indometacin, naproxen, piroxicam, tiaprofen acid, tolfenam acid, paracetamol (acetaminophen) or Rofecoxib. The presence of such agent may be desired, especially when the second component of the device comprises proteolytic enzymes, as the presence of such enzymes in a wound may sometimes give rise to pain for the patient.

Hydrogels are hydrophilic polymer 3-dimensional networks, which have the ability to swell in water or aqueous solvent systems. The polymer network can be established by either physical or chemical interactions. The polymer structure is able to retain the solvents forming a swollen gel phase and especially when cross-linked, will not dissolve but reach an equilibrium swelling level. Hydrogels may absorb water from 10% and up to more than 1000 times their own dry weight. A hydrogel for wound care may therefore have capabilities of providing a moist wound-healing environment and deliver or absorb moisture.

Hydrogel may have a varying degree of viscosity. The hydrogel of the present invention may preferably have a viscosity of 1000 cps to 4.000.000 cps, more preferably 100.000 cps to 2.000.000 cps, even more preferably 500.000 to 1.000.000 cps. In an especially preferred embodiment of the invention the hydrogel has a viscosity of 600.00 cps to 800.000 cps.

In a preferred embodiment of the invention, the hydrogel is of the type as described in European Patent No. 928 206.

In another embodiment of the present invention the hydrogel comprises of one or more of the following polymers, sodiumcarboxymethylcellulose (NaCMC), calciumcarboxymethylcellulose (CaCMC), carboxymethyl cellulose (CMC), hydroxyethylcellulose (HEC), hydroxypropylcellulose (HPC), ethyl cellulose (EC), hydroxypropylmethylcellulose (HPMC), polyvinylpyrrolidone (PVP), polyvinylacetate, polyethyleneglycol (PEG), polyethylene oxide (PEO), alginic acid, pectin, carrageean, chondroitin sulphate, dextran sulphate, chitosan, polylysine, collagen, carboxymethyl chitin, fibrin, dextran, agarose, pullulan, cyclodextrine, p-dioxane, ethylene glycol dimethacrylate, hyaluronic acid, hydroxyethyl methacrylate, poly(acrylic acid), polyethylmethacrylate. Different derivatives, molecular weights, grades, degree of substitution cross-linking rates and agents or interpenetration networks may be used.

In one embodiment the second component is an active ingredient, e.g. a therapeutically or biologically agent.

The active ingredient(s) may be in any suitable physical form, e.g. in the form of a solution, a suspension, a paste or in the form of powder or particles.

In a preferred embodiment the active ingredient may be in the form of a powder or particles.

The second component may be in the form of a dispersion or solution. The particles may be suspended into a carrier-liquid or paste, such as a liquid being a non-solvent to the agent. Examples of such carriers may be oils, surfactants or waxes or mixtures thereof. Having the second component in the form of a liquid or paste may ease the mixing of the components.

The second component may comprise one or more proteins or peptides.

In a preferred embodiment of the invention, the active ingredient may be an enzyme. The enzyme may show enzymatic activity when contacted with wound exudates. The enzyme may preferably be a proteolytic enzyme for debridement of necrotic tissue and slough in a wound. The enzyme may be any suitable enzyme known to be capable of debriding a wound, e.g. papain, collagenase, sutilains, protinase-K, bromelain, trypsin, subtilisin or vibralysin.

The enzyme is preferably in a physical form where it is stable for several years at room temperature. It could be either as a powder, lyophilized powder, encapsulated in protecting polymers or in an appropriate solution/suspension.

The device according to the present invention comprises means for bringing the hydrogel and the active ingredient into contact with each other. In one embodiment of the invention the means for bringing the hydrogel and the active ingredient into contact comprises a rupturable membrane and means for breaking said membrane.

The container may further comprise a third compartment wherein the first and the second component are brought into contact with each other. This compartment may serve as a mixing chamber for the components.

In one embodiment of the invention the means for bringing the first and the second component into contact is a screw. When the components are moved through the screw they are mixed thoroughly.

The container may be prepared from any suitable material. It may be advantageous if the container is prepared from a flexible material. This renders it possible to manipulate with the content of the container, e.g. by squeezing or kneading the container in order to mix the components or to bring the components into contact with each other.

In one embodiment of the invention the device is in the form of a flexible container wherein a second or multiple breakable containers of are enclosed. When pressure is applied to the breakable containers, the contents of these containers will be released to the surrounding component of the first container and the combined components may be mixed by kneading the container.

In one embodiment the container has an accordion-like portion forming a bellow, which may be compressed in order to squeeze the components out of the container, and optionally mixing them at the same time. A barrier layer may separate the two components from each other. The barrier layer may e.g. be in the form of a film or a rupturable wall. When pressure is applied to the bottom of the container, an overpressure is created; hereby the barrier breaks and the two components are joined. By the addition of further pressure, the joined components are moved towards the tip of the container and out of it. The tip of the container may be provided with a screw in order to mix the components more thoroughly before leaving the container.

In order to facilitate the breaking of the barrier layer an internal spear or needle may be placed on the inside of the container. When the accordion-like portion of the container is squeezed the spear will perforate the barrier layer.

In another embodiment of the invention the container is in the form of a flexible bag, which may be kneaded in order to mix the components.

The container may be prepared from rigid or semi-rigid material. The container may e.g. be in the form of a syringe with a piston, the piston may be activated in order to squeeze the components out of the container and optionally mix them.

The compartments of the device of the present invention may be approximately the same size. This will render it easier to obtain a homogenously blending of the components.

In another embodiment of the invention the compartments have different sizes, thus the volume of the first component may be larger than the volume of the second component. Typically, the first compartment, containing the hydrogel is large, while the second compartment, housing the active ingredient, is smaller. The volume ratio between the hydrogel and the second component is preferably in the range of 1000:1 (v/v) to 1:1 (v/v), more preferred 100:1 (v/v) to 5:1 (v/v) and even more preferred 25:1 (v/v) to 10:1 (v/v).

In one embodiment of the invention the volume of the first component is at least the double of the volume of the second component.

The invention further relates to a process for preparing a medicated hydrogel by providing a multi component wound care device comprising a hydrogel and at least one more components placed in separate compartments of a container, bringing the two or more components into contact with each other, and mixing said components.

The two or more components may be mixed by passing a screw.

The means for bringing the components into contact may be by breaking a rupturable membrane.

Even though the invention is mostly described for a two-component system, it is obvious that a multi-component system would work under the same principles. E.g. instead of mixing of two components a three-component system with three separate compartments may be prepared. An additional component, e.g. a surfactant such as urea may also be present, or additional enzymes.

The device of the present invention is in the form of a single unit. This is advantageous compared to devices comprising multiple units being assembled before use. Having only one unit, everything is present in one device, as well as the risk of leakage and contamination is negligible.

The container and the means for bringing the components into contact with each other may be constructed in such a way the components may be added to the mixture one by one, thus the individual compartments of the container may be opened independent of the other compartments. In this way a tailor made device may be prepared.

In one embodiment of the present invention the compartment comprising the active ingredient may be provided with a dosimeter rendering it possible to add a desired amount of active ingredient to the hydrogel.

### DESCRIPTION OF THE PREFERRED EMBODIMENTS

The invention is now explained more in detail with reference to the drawings showing preferred embodiments of the invention.

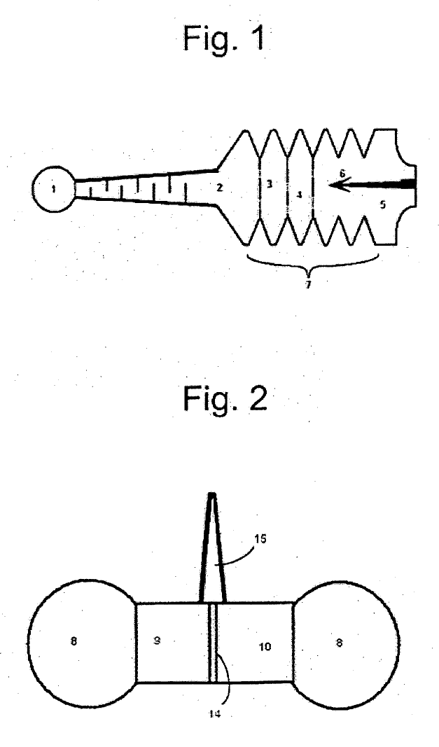

In FIG. 1 is shown an embodiment of the invention having the shape of an accordion (7) and having a spear activator (6). The device further comprises a tip (1), an application nozzle with build-in static mixer (2), separated compartments for hydrogel (5) and active ingredients (3,4). The compartments (3, 4, 5) may be arranged in other ways than by vertical separation, and the numbers of compartments may vary. The compartments are separated from each other by membranes. When preparing the device for use, the tip (1) is torn off. A physical pressure is applied to the bottom of the device, thus compressing the accordion-like portion (7). Hereby the spear (6) penetrates the membranes, and by application of further pressure, the components of the compartments (3, 4, 5) are pressed through the nozzle (2) with the build-in static mixer and thus mixed before leaving the tip of the device for application.

In FIG. 2 is shown another embodiment of the invention having two air-filled bubbles (8) next to two separate compartments (9, 10) for a hydrogel and for an active ingredient. The compartments are separated from each other by a breakable membrane (14). The device further comprises a nozzle (15). When preparing the device for use, one or both of the air-filled bubbles (8) are subjected to physical pressure. Due to increased pressure the membrane (14) breaks and the components are united. The components can by thoroughly mixed by pressing each air bubble (8) in turn. When sufficient mixing of the components is obtained, both air-filled bubbles (8) are compressed and the united components of the compartments are pressed through the nozzle (15) of the device.

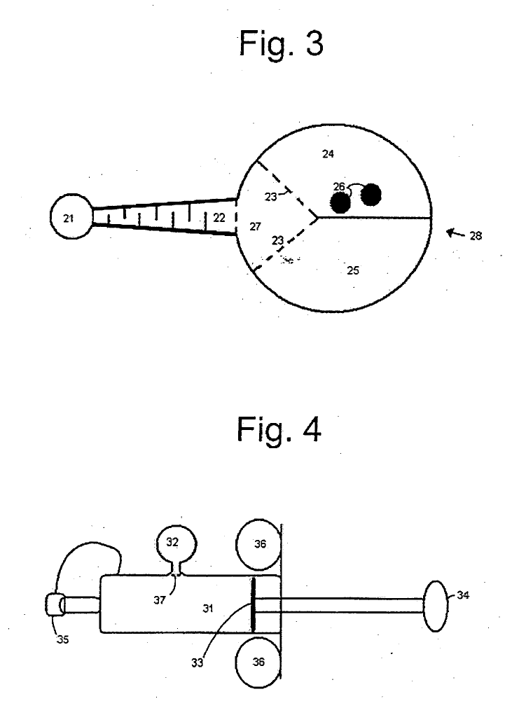

FIG. 3 shows a third embodiment of the invention having at one end a flexible ball (28) comprising three separate compartments for a hydrogel (25), an active ingredient (24) and for mixing of the components (27). Compartment (24) further comprises mixing balls (26) for facilitating the mixing of the components. The compartment for mixing (27) is separated from the compartments (24.25) by two membranes (23). In connection with the compartment for mixing (27) is a nozzle with build-in static mixer (22) leading to a tip (21). The tip (21) may be broken off before use.

When the ball (28) is squeezed the membranes (23) break and the components are united. By kneading the ball (28) the united components are blended into a substantially homogenous mixture with help from the mixing balls (26). Thereafter the tip (21) is broken off and the mixture is pressed out through the nozzle (22), which has a build-in static mixer for further mixing.

FIG. 4 shows yet another embodiment of the invention in the form of a syringe. The device has a compartment for a hydrogel (31), a compartment for an active ingredient (32) separated from the first compartment by a membrane (37), a piston (33) connected to a rod (34) having two handles for fingers (36) and a tip equipped with a cap (35). The bubble-like compartment (32) is subjected to physical pressure, which causes the membrane to break, and the active ingredient is introduced into the larger compartment (31) containing the hydrogel. By moving the piston (33) up and down the two components are mixed. Then the cap (35) is removed and the mixture is pressed out.

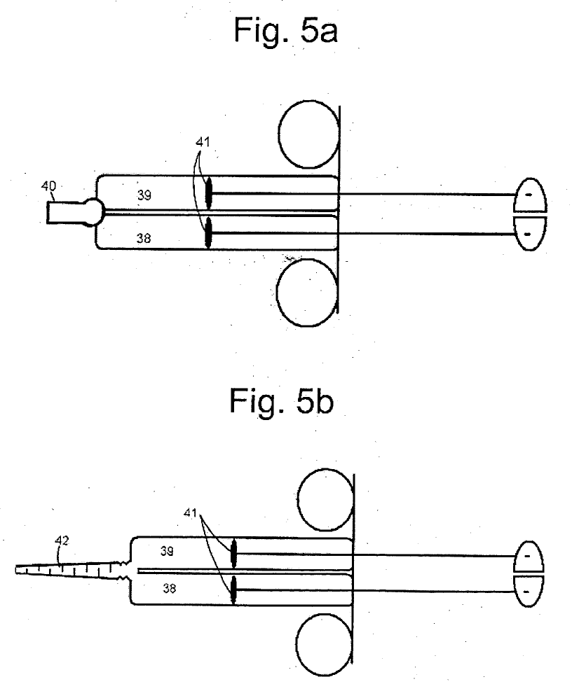

FIGS. 5a and 5b shows an embodiment of the invention in the form of a 2-chamber syringe. The device has a compartment for a hydrogel (38), a compartment for an active ingredient (39) separated from the first compartment by the cap (40), a 2-part piston (41). When the cap is removed and replaced by a mixer head/nozzle (42) there is open between the 2 compartments. Buy moving the two pistons up and down in different directions the components are mixed. By moving the pistons simultaneously down the mixture is pressed out through the nozzle.

### EXAMPLES

A hydrogel as in European Patent No. 928 206 was prepared and placed in one compartment of a device as shown in FIG. 5. A papain preparation as shown below was placed in the other compartment. The device was radiated for sterility. After 3 months activity of the enzyme was measured. A test was performed in order to evaluate the conformity of mixing. The mixing was initiated and the mixture was pressed out in a 50 cm long band. 1 cm samples were taken from start, middle and end. The samples were weighed, diluted in 50 ml water and the protein content and enzyme activity was measured.

Using 15 gr. of hydrogel in compartment 1, this was done with the following papain vehicles in compartment 2:
1. 1 g of microgranular papain preparation as a spray-dried form with the carrier, maltodextrine facilitating fast dissolution.
2. A suspension comprising 1 gr. of a microgranular papain powder in 2 ml of Tween 80. Instead of Tween 80 another nonionic surfactants may be used, e.g. various grades of Brij, Cremophor, Genapol, Pluronic, Triton or Tween.
3. A suspension consisting of 1 gr. of a microgranular papain powder in 2 ml of the hydrophobic vehicle, 10% w/w polyethylene and 90% w/w liquid paraffin, Ph.Eur. Another lipophilic vehicle may be used, e.g. various grades of ethylolate, glycerolmonosterat, glycerolmonooleat, isopropylmyristat, light mineral oil, polyethyleneglycol 800 and higher (macrogol) or different vegetable oils.
4. 3 ml of a stabilized papain solution of water/sorbitol (50:50) and 1 gr. of pure papain. Instead of water/sorbitol other components could be in the solution, e.g. glycerol, sucrose, propyleneglycol, polyethyleneglycol 200-600 (macrogol).

The results showed that during sterilization and storage there was no loss in activity. In the mixing studies no significant differences between the samples were measured.

1. A wound care device comprising a container having two or more separate compartments, a first compartment containing a first component, a second compartment containing a second component, wherein the container comprises means for bringing the first component into direct contact with the second component, and means for mixing the components and wherein the first component is a hydrogel.

2. A device according to claim 1, wherein the second component comprises one or more active ingredients.

3. A device according to claim 1, wherein the second component is in the form of powder or particles.

4. A device according to claim 1, wherein the second component is in the form of a dispersion or solution.

5. A device according to claim 1, wherein the second component comprises one or more proteins.

6. A device according to claim 1, wherein the second component comprises one or more enzymes.

7. A device according to claim 6, wherein the enzyme is selected from the group of papain, collagenase, sutilains, protinase-K, bromelain, trypsin, subtilisin or vibralysin.

8. A device according to claim 1, wherein the volume of the first component is larger than the volume of the second component.

9. A device according to claim 1, wherein the volume of the first component is at least the double of the volume of the second component.

10. A device according to claim 1, wherein the means for bringing the first and the second component into contact comprises a rupturable membrane and means for breaking said membrane.

11. A device according to claim 1, wherein the container further comprises a third compartment wherein the first and the second component are brought into contact with each other.
